(19) United States
(12) Patent Application Publication
(10) Pub. No.: US 2025/0357006 A1
(43) Pub. Date: Nov. 20, 2025

(54) METHOD AND APPARATUS PREDICTING OBSTRUCTIVE SLEEP APNEA

(71) Applicants: AJOU UNIVERSITY INDUSTRY-ACADEMIC COOPERATION FOUNDATION, GYEONGGI-DO (KR); Dankook University Cheonan Campus Industry Academic Cooperation Foundation, Chungcheongnam-do (KR)

(72) Inventors: Kim TAE-JOON, Gyeonggi-do (KR); Shin HYE-RIM, Chungcheongnam-do (KR); Park JUNE-YOUNG, Gyeonggi-do (KR); Kim YUNSOO, Gyeonggi-do (KR); Kim MIN HYE, Gyeonggi-do (KR)

(73) Assignees: AJOU UNIVERSITY INDUSTRY-ACADEMIC COOPERATION FOUNDATION, GYEONGGI-DO (KR); Dankook University Cheonan Campus Industry Academic, Chungcheongnam-do (KR)

(21) Appl. No.: 19/211,065

(22) Filed: May 16, 2025

(30) Foreign Application Priority Data
May 17, 2024 (KR) 10-2024-0064573

(51) Int. Cl. G16H 50/20; G16H 10/20; G16H 50/30

(52) U.S. Cl. CPC G16H 50/20; G16H 10/20; G16H 50/30

(57) ABSTRACT

The present disclosure relates to a method and apparatus for predicting obstructive sleep apnea. The method for predicting obstructive sleep apnea according to one embodiment of the present disclosure includes generating analysis data from facial photograph information of an analysis subject, storing response data of an OSA screening questionnaire of the analysis subject in the memory, inputting the analysis data and the response data into a pre-trained machine learning model and inferring information about the degree of OSA, and transmitting the inference result to at least one terminal or outputting the inference result to a display.

### CROSS-REFERENCE TO RELATED APPLICATION

This application claims priority to and the benefit of Korean Patent Application No. 10-2024-0064573, filed on May 17, 2024, the disclosures of which is incorporated herein by reference in its entirety.

### BACKGROUND

The present disclosure relates to a technique for predicting obstructive sleep apnea (OSA), and more specifically, to a technique for predicting the degree of OSA of a subject based on answers to an OSA screening questionnaire and analysis result of a facial photograph.

Obstructive sleep apnea (OSA) is a sleep-related breathing disorder in which an airway is repeatedly blocked during sleep, temporarily stopping breathing, and may be caused by genetics, aging, obesity, enlarged tonsils, or the like. In particular, the OSA may cause hypertension, heart attack, stroke, memory loss, depression, or sleep-related gastrointestinal disorder.

Polysomnography (PSG) is essential for an accurate diagnosis of the OSA, but there are inconveniences due to wearing the equipment and testing overnight. Accordingly, OSA screening questionnaires such as Berlin, STOP, or STOP-BANG have been developed for the initial screening of OSA. However, a technique (hereinafter, referred to as a “first technique”) for diagnosing OSA using these OSA screening questionnaires is only a technique for roughly diagnosing OSA based on answers of a subject to the OSA screening questionnaire, and therefore has a problem in that the accuracy of OSA diagnosis is low, at approximately 69% to 73.8%.

Meanwhile, anatomical information about a craniofacial region of the subject or the like, may correspond to factors associated with the OSA. Accordingly, a technique (hereinafter, referred to as “second technique”) for predicting the OSA using a machine learning model trained based on a facial photograph of the subject has been proposed. However, since this second technique simply performs prediction of the OSA based on only the subject's facial photograph, there is a problem in that the accuracy of the prediction does not reach a certain level (for example, 85% or higher).

However, the above-described contents and the first and second technologies merely provide background information for the present disclosure and do not correspond to previously disclosed technologies.

### SUMMARY

In order to solve the problems of the above-described prior art, an object of the present disclosure is to provide a technique for more accurately predicting the degree of obstructive sleep apnea (OSA) for a subject by comprehensively using a subject's answer to a screening questionnaire of the OSA and analysis results of a facial photograph.

However, an object of the present disclosure is not limited to the object above, and other objects not mentioned can be clearly understood by a person having ordinary knowledge in the technical field to which the present disclosure belongs from the description below.

In order to achieve the above-described objects, according to one embodiment of the present disclosure, there is provided a method for predicting obstructive sleep apnea (OSA), the method including: generating analysis data from facial photograph information of an analysis subject; storing response data of an OSA screening questionnaire of the analysis subject in the memory; inputting the analysis data and the response data into a pre-trained machine learning model and inferring information about the degree of OSA; and transmitting the inference result to at least one terminal or outputting the inference result to a display.

In one embodiment of the present disclosure, the generating of the analysis data may include inputting the facial photograph information into a first machine learning model, and generating first analysis data inferring the degree of OSA by the first machine learning model.

In one embodiment of the present disclosure, the inferring of the information about the degree of OSA may include inputting the first analysis data and the response data into a second machine learning model, and inferring the information about the degree of OSA by the second machine learning model and generating the inference result.

In one embodiment of the present disclosure, the generating of the analysis data may include extracting a plurality of landmark information from the facial photograph information, and generating second analysis data, which is distance information between the landmarks, by using the plurality of landmark information.

In one embodiment of the present disclosure, the inferring of the information about the degree of OSA may include inputting the second analysis data and the response data into a third machine learning model, and inferring information about the degree of OSA by the third machine learning model and generating the inference result.

In one embodiment of the present disclosure, the generating of the analysis data may include inputting the facial photograph information into a first machine learning model, generating first analysis data inferring the degree of OSA by the first machine learning model, extracting a plurality of landmark information from the facial photograph information, and generating second analysis data, which is distance information between the landmarks, using the plurality of landmark information, and the inferring of the information about the degree of OSA may include inputting the first analysis data, the second analysis data, and the response data into a fourth machine learning model, and inferring the information about the degree of OSA by the fourth machine learning model and generating the inference result.

In one embodiment of the present disclosure, the machine learning model may infer information about a plurality of classes based on a preset range for an apnea-hypopnea index or a respiratory distress index.

In order to achieve the above-described objects, according to one embodiment of the present disclosure, there is provided an apparatus for predicting obstructive sleep apnea (OSA), the apparatus including: a memory that stores at least one instruction; a processor that executes at least one instruction, in which the processor may generate analysis data from facial photograph information of an analysis subject, store response data of an OSA screening questionnaire of the analysis subject in the memory, input the analysis data and the response data into a pre-trained machine learning model and infer information about the degree of OSA, and transmit the inference result to at least one terminal or output the inference result to a display.

In one embodiment of the present disclosure, the processor may input the facial photograph information into a first machine learning model, and generate first analysis data inferring the degree of OSA by the first machine learning model to generate the analysis data.

In one embodiment of the present disclosure, the processor may input the first analysis data and the response data into a second machine learning model, and infer the information about the degree of OSA by the second machine learning model and generate the inference result to infer the information about the degree of OSA.

In one embodiment of the present disclosure, the processor may extract a plurality of landmark information from the facial photograph information, and generate second analysis data, which is distance information between the landmarks, by using the plurality of landmark information to generate the analysis data.

In one embodiment of the present disclosure, the processor may input the second analysis data and the response data into a third machine learning model, and infer information about the degree of OSA by the third machine learning model to infer the information about the degree of OSA.

In one embodiment of the present disclosure, the processor may input the facial photograph information into a first machine learning model, generate first analysis data inferring the degree of OSA by the first machine learning model, extract a plurality of landmark information from the facial photograph information, generate second analysis data, which is distance information between the landmarks, using the plurality of landmark information, to generate the analysis data, input the first analysis data, the second analysis data, and the response data into a fourth machine learning model, and infer the information about the degree of OSA by the fourth machine learning model and generate the inference result to infer the information about the degree of OSA.

In one embodiment of the present disclosure, the machine learning model may infer information about a plurality of classes based on a preset range for an apnea-hypopnea index or a respiratory distress index.

According to the present disclosure configured as described above, the degree of OSA of a subject is predicted by comprehensively reflecting the subject's answers to the OSA screening questionnaire and the analysis results of a facial photograph containing anatomical information, and thus, it is possible to increase the accuracy of the prediction.

In addition, according to the present disclosure, the degree of OSA of the subject is predicted by using the second machine learning model trained based on the answers to the OSA screening questionnaire and the analysis results of the first machine learning model for the facial photograph, respectively, and thus, it is possible to predict the OSA information of the subject very accurately.

In addition, according to the present disclosure, the degree of OSA of the subject is predicted by using the third machine learning model trained based on the answers to an OSA screening questionnaire and the distance to landmarks of the facial photograph, respectively, and thus, it is possible to predict the OSA information of the subject very accurately.

The effects that can be obtained from the present disclosure are not limited to the effects mentioned above, and other effects that are not mentioned can be clearly understood by those skilled in the art from the description below.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF THE DRAWING

The above and other aspects, features and other advantages of the present disclosure will be more clearly understood from the following detailed description taken in conjunction with the accompanying drawings, in which:

FIG. 1 illustrates a schematic block diagram of an electronic device according to one embodiment of the present disclosure;

FIG. 2 illustrates an example of a landmark in a facial photograph;

FIG. 3 illustrates a flowchart of a method according to one embodiment of the present disclosure; and

FIG. 4 illustrates a result graph for a first experiment on performance comparison between one embodiment of the present disclosure and a comparative example.

### DETAILED DESCRIPTION

The purpose and means of the present disclosure and the effects thereof will become clearer through the following detailed description related to the attached drawings, and accordingly, those skilled in the art can easily carry out the technical idea of the present disclosure. In addition, when describing the present disclosure, if it is determined that a specific description of a known technique related to the present disclosure may unnecessarily obscure the gist of the present disclosure, the detailed description will be omitted.

The terms used in the present specification are for the purpose of describing embodiments and are not intended to limit the present disclosure. In the present specification, the singular includes the plural as well, unless specifically stated in the phrase. In the present specification, the terms “include”, “provide”, “provide”, or “have” do not exclude the presence or addition of one or more other components other than the mentioned components.

In the present specification, the terms “or”, “at least one”, or the like may represent one of the words listed together, or may represent a combination of two or more. For example, “A or B”, “at least one of A and B” may include only one of A or B, or may include both A and B.

In the present specification, the description using the word “for example” or the like should not be construed as limiting the embodiments of the disclosure by the effect of variations such as tolerances, measurement errors, limitations of measurement accuracy, and other commonly known factors, as well as the information presented, such as cited characteristics, variables, or values, may not be exactly the same.

In the present specification, when a component is described as being “coupled” or “connected” to another component, it should be understood that it may be directly connected or connected to the other component, but there may be another component in between. Meanwhile, when a component is described as being “directly coupled” or “directly connected” to another component, it should be understood that there is no other component in between.

In the present specification, when a component is described as being “on” or “in contact with” another component, it should be understood that it may be directly on or connected to the other component, but there may be another component in between. Meanwhile, when a component is described as being “directly on” or “in direct contact with” another component, it should be understood that there is no other component in between. Other expressions that describe the relationship between components, such as “between” and “directly between”, may be interpreted in the same way.

In the present specification, the terms “first”, “second”, or the like. may be used to describe various components, but the components should not be limited by the terms. In addition, the terms should not be construed as limiting the order of each component, and may be used for the purpose of distinguishing one component from another. For example, a “first component” may be named the “second component”, and similarly, the “second component” may also be named the “first component”.

Unless otherwise defined, all terms used in the present specification may be used in a meaning that can be commonly understood by a person having ordinary knowledge in the technical field to which the present disclosure belongs. In addition, terms defined in a commonly used dictionary shall not be ideally or excessively interpreted unless explicitly specifically defined.

Hereinafter, preferred embodiments according to the present disclosure will be described in detail with reference to the attached drawings.

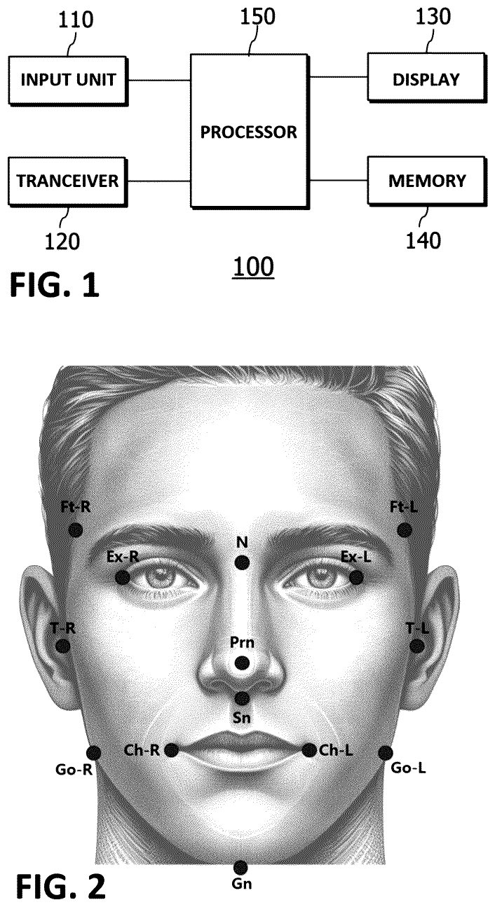

FIG. 1 illustrates a schematic block diagram of an electronic device 100 according to one embodiment of the present disclosure.

The electronic device 100 (hereinafter, referred to as the “present device”) according to one embodiment of the present disclosure is a device that performs a prediction of the degree of obstructive sleep apnea (OSA). That is, the present device 100 may predict the degree of OSA for the subject. In particular, the present device 100 may predict the current degree of OSA for the subject by using subject's answers to an OSA screening questionnaire and analysis results of a subject's facial photograph.

To this end, the present device 100 may train a machine learning model, which will be described later, using training data prepared based on multiple test-subjects, and then predict the degree of OSA for a subject by using each trained machine learning model. That is, the test-subject is a person who provides various data necessary to prepare training data. Meanwhile, the subject is a person who is the subject of prediction of the degree of OSA according to the present disclosure, and is the subject of performing inference on the trained machine learning model.

In this case, the degree of OSA indicates the degree of the state of OSA, and may include multiple states. For example, the degree of OSA may include four states: normal, mild degree, moderate degree, and severe degree. In this case, normal is a general state in which the test-subject or the subject is determined or expected not to have OSA symptoms, and the mild degree, moderate degree, and severe degree are states in which the test-subject or the subject is determined or expected to have OSA symptoms. Of course, it means that the OSA symptoms become more severe as the degree goes from the mild degree to the moderate and severe degrees. Moreover, the degree of OSA may simply include the first and second states, as will be described later.

Of course, the degree of OSA may be divided into multiple states according to the range of the apnea-hypopnea index (AHI) or the range of the respiratory distress index (RDI). In this case, the AHI or RDI is an index that can be derived as a result of polysomnography (PSG), and a higher value of the AHI or RDI means that the OSA symptoms are more severe. At this time, each state of the degree of OSA may have a different AHI range or a different RDI range according to the reference value for the AHI or RDI, and there may be at least one reference value for the AHI or RDI.

For example, there may be first to third reference values for the AHI or RDI. In this case, normal may correspond to a case having an AHI or RDI range that is less than the first reference value, and the mild degree may correspond to a case having an AHI or RDI range that is greater than or equal to the first reference value and less than the second reference value. In addition, the moderate degree may correspond to a case having an AHI or RDI range that is greater than or equal to the second reference value and less than the third reference value, and the severe degree may correspond to a case having an AHI or RDI range that is greater than or equal to the third reference value.

For example, the AHI or RDI of the first reference value may be 5, the AHI or RDI of the second reference value may be 15, and the AHI or RDI of the third reference value may be 30. In this case, the normal may correspond to the case where AHI<5 or RDI<5, the mild degree may correspond to the case where 5≤AHI<15 or 5≤RDI<15, the moderate degree may correspond to the case where 15≤AHI<30 or 15≤RDI<30, and the severe degree may correspond to the case where 30≤AHI or 30≤RDI.

Of course, the degree of OSA may include more or fewer states than the four states of the normal, mild degree, moderate degree, and severe degree depending on the AHI or RDI range. In particular, it may be desirable for the degree of OSA to include fewer states than the four states, because when the degree of OSA has four states or more than the four states, the accuracy of the first to fourth machine learning models described below may decrease, or the medically and clinically important degrees and the degrees requiring treatment may be different from each other.

Accordingly, the degree of OSA may simply include the first and second states rather than the four states. In this case, the first state may be a state in which the test-subject or subject is determined or expected to have no OSA symptom or mild OSA symptoms. Meanwhile, the second state is a state in which the test-subject or subject is determined or expected to have moderate or severe OSA symptoms.

In this case, there may be one reference value for the AHI or RDI. Accordingly, the first state may correspond to a case in which the AHI or RDI range is smaller than the reference value, and the second state may correspond to a case in which the AHI or RDI range is greater than the reference value. Of course, when the AHI or RDI range corresponds to the reference value, the degree of OSA may correspond to either the first or second state.

When there is one reference value for the AHI or RDI, the reference value may be a value of AHI or RDI between 5 and 15, preferably a value of 15, but is not limited thereto. For example, when the reference value is 15, the first state may correspond to a case where the AHI or RDI range is less than 15, and the second state may correspond to a case where the AHI or RDI range is greater than 15. Of course, when the AHI or RDI range corresponds to 15, the degree of OSA may correspond to either the first or second state. However, the present disclosure is not limited thereto, and the first reference value may have a value other than 15.

Meanwhile, the OSA screening questionnaire is a questionnaire used for screening OSA and includes a number of questions related to OSA. Each answer of the test-subject to a number of questions according to the OSA screening questionnaire may be generated as response data and may be included in the input data of the training data for the second to fourth machine learning models described below. For example, the OSA screening questionnaire can be Berlin, STOP, or STOP-BANG, but is not limited thereto.

Berlin is one of the most commonly used screening tools for diagnosing OSA in various countries including Korea. This Berlin consists of three categories of questions on OSA-related symptoms. Here, Category 1 includes five questions on “whether snoring”, “snoring intensity”, “snoring frequency”, “whether snoring causes disturbance”, and “whether apnea or choking is witnessed during sleep”. Category 2 includes four questions on “whether fatigue after sleep”, “whether fatigue upon awakening”, “whether drowsy driving”, and “drowsy driving frequency”. Category 3 includes questions about “whether to have hypertension” and “whether BMI exceeds a certain value (for example, 30 kg/m²)”. For these questions, the test-subject or subject answers “yes” or “no” to the “whether” question, or answers the corresponding intensity to the “intensity” question.

STOP includes four questions in total about “snoring”, “daytime fatigue”, “witnessing apneas or choking during sleep”, and “hypertension”. The test-subject or subject answers “yes” or “no” to these questions.

STOP-Bang includes four additional questions in addition to the four questions included in STOP, for a total of eight questions. Namely, in STOP-Bang, four questions are added about “whether BMI exceeds a certain value (for example, 35 kg/m²)”, “whether age exceeds a certain value (for example, 50 years old)”, “whether neck circumference exceeds a certain value (for example, 40 cm)”, and “whether gender is male”. The test-subject or subject answers “yes” or “no” to these questions.

For example, the OSA screening questionnaire used in the present device 100 may include a combination of questions included in Berlin or questions included in STOP-BANG. That is, the OSA screening questionnaire may include only questions included in Berlin or only questions included in STOP-BANG. Alternatively, the OSA screening questionnaire may include at least one question included in Berlin and at least one question included in STOP-BANG.

Of course, it may be desirable for the OSA screening questionnaire to include questions included in STOP-BANG. This is because STOP-BANG is composed of questions that can be answered more easily by the test-subject or the subject, while at the same time being composed of questions of various categories related to OSA. Of course, the present disclosure is not limited thereto, and the OSA screening questionnaire may include the questions included in Berlin or questions of other types of OSA screening questionnaires in addition to the questions of STOP-BANG.

The first state and the second state, which are labels based on the OSA screening questionnaire, may be classified according to the STOP-BANG score. For example, the STOP-BANG score may be 3, 4, or 5. The STOP-BANG score is not limited to the above example and may be set to other values.

The OSA screening questionnaire may be stored in a memory 140. The OSA screening questionnaire may be transmitted to a user terminal through a tranceiver 120, and a survey subject may input the answer so that the user terminal can generate and store the response data. The response data may be transmitted from the user terminal to the present device 100, and the tranceiver 120 may provide the response data to a processor 150. The response data may also be generated by a method in which the user of the device 100 directly inputs the survey subject's answer through an input unit 110.

However, when the degree of OSA is predicted using only the answer to the OSA screening questionnaire, the accuracy is bound to be low. Accordingly, the present device 100 predicts the degree of OSA of the subject by further using the analysis results of a facial photograph of the test-subject or the subject in addition to the answer to the OSA screening questionnaire.

In this case, the facial photograph of the test-subject or subject includes anatomical information about the subject's craniofacial abnormality, or the like, and for the analysis thereof, the present device 100 may use the first machine learning model (hereinafter, referred to as “first model”) to be described later, or may use the distance to a landmark in the facial photograph of the test-subject or subject. That is, the analysis result of the facial photograph of the test-subject or subject may be data (hereinafter, referred to as “first analysis data”) related to the result of performing inference on the first model, or may be data (hereinafter, referred to as “second analysis data”) related to the distance to the landmark in the facial photograph of the test-subject or subject. Accordingly, the first analysis data or the second analysis data may be included in the input data of the training data for the second to fourth machine learning models to be described later.

The first model is a model trained according to the machine learning technique of supervised learning using the first training data. That is, the first model is a classification model that predicts a plurality of classes for various ranges of AHI or RDI of the test-subject or subject when the facial photograph of the test-subject or subject is input. In this case, each class may include a range for different AHI or RDI.

For example, the classes predicted by the first model may include each class for the four states described above. In this case, the normal class may be a class for the range of AHI or RDI corresponding to the normal degree of OSA. The mild class may be a class for the range of AHI or RDI corresponding to the mild degree of OSA. The moderate class may be a class for the range of AHI or RDI corresponding to the moderate degree of OSA. The severe class may be a class for the range of AHI or RDI corresponding to the severe degree of OSA.

Of course, in order to further increase accuracy, the class predicted by the first model may include each class for the first and second states described above. In this case, the first class may be a class for the range of AHI or RDI corresponding to the first state. The second class may be a class for the range of AHI or RDI corresponding to the second state.

Accordingly, the first training data, which is data required to train the first model, includes a pair of input data and output data. In this case, the input data of the first training data may include the facial photograph of the test-subject. Of course, it may be preferable that the facial photograph of the test-subject be a frontal or side facial photograph of the subject. This is because the frontal or side photograph may include more anatomical information about the test-subject's craniofacial abnormalities, or the like. Of course, the facial photograph is a digital photo according to pixels that can be stored on a computer, and may be in a photo format such as JPG, but is not limited thereto.

In addition, the output data of the first training data is a label for the facial photograph of the test-subject, which is the input data. The output data of this first training data may include labels for the aforementioned plurality of classes. That is, the label for which class the value of AHI or RDI derived from the PSG result for the test-subject belongs may be included as the output data.

Of course, the first training data may include pairs of input data and output data for multiple different test-subjects. According to the learning using this first training data, the first model has a function for the relationship between the input data of the first training data and the output data of the training data, and expresses the function using various parameters. That is, the first model may express the relationship between the input data and the output data of the first training data using parameters of weights and biases.

Accordingly, when inference is performed on the first model trained with the first training data by inputting the facial photograph of the test-subject or subject as the input data, the first model outputs the probability value of each class for the test-subject or subject as the output data as a result. In this case, the corresponding probability value of the class with the largest probability value among the probability values of each class or the processed value of the corresponding probability value may be utilized as the first analysis data. In this case, the processed value may be the result value of performing a specific operation on the corresponding probability value. For example, the specific operation may include an operation of multiplying, adding, or dividing the corresponding probability value by a specific value determined in advance, but is not limited thereto.

The second machine learning model (hereinafter, referred to as the “second model”) is a model trained according to the machine learning technique of supervised learning using the second training data. In other words, the trained second model is a model that predicts the degree of OSA of the subject when the response data for the OSA screening questionnaire of the subject and the first analysis data of the subject are input.

In this case, the degree of OSA predicted by the second model may include the four states described above. Of course, in order to further increase accuracy, the degree of OSA predicted by the second model may include the first and second states described above.

Accordingly, the second training data, which is the data required to train the second model, includes pairs of input data and output data. In this case, the input data of the second training data may include the response data for the test-subject's OSA screening questionnaire and the test-subject's first analysis data. In addition, the output data of the second training data is a label for the corresponding input data (that is, the response data for the test-subject's OSA screening questionnaire and the test-subject's first analysis data). The output data of this second training data may include a label for the degree of OSA described above.

Of course, the second training data may include pairs of input data and output data for the multiple different test-subjects. According to the learning using the second training data, the second model has a function for the relationship between the input data of the second training data and the output data of the training data, and expresses the function using various parameters. That is, the second model may express the relationship between the input data and the output data of the second training data using the parameters of weights and biases.

The third machine learning model (hereinafter, referred to as the “third model”) is a model trained according to the machine learning technique of supervised learning using the third training data. That is, the trained third model is a model that predicts the degree of OSA of the subject when the response data for the OSA screening questionnaire of the subject and the second analysis data of the subject are input.

In this case, the degree of OSA predicted by the third model may include the four states described above. Of course, in order to further increase the accuracy, the degree of OSA predicted by the third model may include the first and second states described above.

Accordingly, the third training data, which is the data required to train the third model, includes a pair of input data and output data. In this case, the input data of the third training data may include the response data for the OSA screening questionnaire of the test-subject and the second analysis data.

FIG. 2 illustrates an example of the landmark in the facial photograph.

The second analysis data include the distance to the landmark in the facial photograph of the test-subject or subject. In this case, it may be desirable for the facial photograph of the test-subject or subject to include a frontal photograph and a side photograph of the face of the test-subject or subject, respectively.

As an example, referring to FIG. 2, landmarks in the frontal and side facial photographs of the test-subject or subject may be represented as illustrated in Table 1 below.

TABLE 1

| Landmark | Indication |
|---|---|
| Forehead right end point | Ft-R |
| Forehead left end point | Ft-L |
| Right exocanthion of right eye | Ex-R |
| Left exocanthion of left eye | Ex-L |
| Right tragion | T-R |
| Left tragion | T-L |
| Nasion | N |
| Maximum protrusion point of nose | Prn |
| Subnasion | Sn |
| Right chelion | Ch-R |
| Left chelion | Ch-L |
| Mandibular point | Gn |
| Right gonion | Go-R |
| Left gonion | Go-L |

That is, the landmarks may include at least one of the forehead right end point Ft-R, the forehead left end point Ft-L, the right exocanthion of right eye Ex-R, the left exocanthion of left eye Ex-L, the right tragion T-R, the left tragion T-L, the nasion N, the maximum protrusion point of nose Prn, the subnasion Sn, the right chelion Ch-R, the left chelion Ch-L, the mandibular point Gn, the right gonion Go-R, and the left gonion Go-L. Accordingly, the distance to the landmark in the corresponding facial photograph may be expressed as in Table 2 below.

TABLE 2

| Type of distance to landmark | Indication |
|---|---|
| Forehead width | Distance between Ft-R and Ft-L |
| Binocular width | Distance between Ex-R and Ex-L |
| Length from right exocanthion of | Distance between T-R and N |
| right eye to nasion | |
| Length from right exocanthion of left | Distance between T-L and N |
| eye to the nasion | |
| Face width | Distance between T-R and T-L |
| Nose height | Distance between Prn and Sn |
| Length from right tragion to | Distance between T-R and Sn |
| subnasion | |
| Length from left tragion to subnasion | Distance between T-L and Sn |
| Length from right tragion to right | Distance between T-R and Ch-R |
| chelion | |
| Length from left tragion to left | Distance between T-L and Ch-L |
| chelion | |
| Lip width | Distance between Ch-R and Ch-L |
| Mandibular height | Distance between Sn and Gn |
| Length from right exocanthion of | Distance between Ex-R and Go-R |
| right eye to right gonion | |
| Length from left exocanthion of left | Distance between Ex-L and Go-L |
| eye to left gonion | |
| Length from right gonion to left | Distance between Go-R and Go-L |
| gonion | |

That is, the distance to the landmark may correspond to the distance between different landmarks, and there may be a plurality of types. For example, the distance to landmarks may include at least one of the forehead width (distance between Ft-R and Ft-L), the binocular width (distance between Ex-R and Ex-L), the length from the right exocanthion of right eye to the nasion (distance between T-R and N), the length from the right exocanthion of the left eye to the nasion (distance between T-L and N), the face width (distance between T-R and T-L), the nose height (distance between Prn and Sn), the length from the right tragion to the subnasion (distance between T-R and Sn), the length from the left tragion to the subnasion (distance between T-L and Sn), the length from the right tragion to the right chelion (distance between T-R and Ch-R), the length from the left tragion to the left chelion (distance between T-L and Ch-L), the lip width (distance between Ch-R and Ch-L), the mandibular height (distance between Sn and Gn), the length from the right exocanthion of the right eye to the right gonion (distance between Ex-R and Go-R), the length from the left exocanthion of the left eye to the left gonion (distance between Ex-L and Go-L), and the length from the right gonion to the left gonion (distance between Go-R and Go-L). That is, in the facial photograph of the test-subject or the subject, the plurality of different distances to the landmarks includes anatomical information about the facial abnormality of the test-subject or the subject, and thus may correspond to the analysis results for the facial photograph of the test-subject or the subject. Accordingly, the plurality of different distances to the landmarks may be used as the second analysis data for the facial photograph of the test-subject and may be included as the input data of the third training data.

The coordinates of the landmarks may be extracted using a landmark extraction program prepared in advance or an artificial intelligence model trained to extract landmarks from facial photographs. The processor 150 may calculate the distances using the extracted landmark coordinates.

In addition, the output data of the third training data is a label for the corresponding input data (that is, the response data for the OSA screening questionnaire of the test-subject and the second analysis data of the test-subject). The output data of this third training data may include a label for the degree of OSA described above.

Of course, the third training data may include pairs of input data and output data for the multiple different test-subjects. According to the learning using this third training data, the third model has a function for the relationship between the input data of the third training data and the output data of the training data, and expresses the function using various parameters. That is, the third model may express the relationship between the input data and the output data of the third training data using the parameters of weights and biases.

The fourth machine learning model (hereinafter, referred to as the “fourth model”) is a model trained according to the machine learning technique of supervised learning using the fourth training data. This fourth model has the characteristic of including the input data of the second model and the input data of the third model together. That is, the trained fourth model is a model that predicts the degree of OSA of the subject when the response data for the OSA screening questionnaire of the subject, the first analysis data of the subject, and the second analysis data of the subject are input.

In this case, the degree of OSA predicted by the fourth model may include the four states described above. Of course, in order to further increase the accuracy, the degree of OSA predicted by the fourth model may also include the first and second states described above.

Accordingly, the fourth training data, which is the data required to train the fourth model, includes a pair of input data and output data. In this case, the input data of the fourth training data may include the response data for the test-subject's OSA screening questionnaire, the test-subject's first analysis data, and the test-subject's second analysis data. In addition, the output data of the fourth training data is a label for the corresponding input data (that is, the response data for the test-subject's OSA screening questionnaire, the test-subject's first analysis data, and the test-subject's second analysis data). The output data of the fourth training data may include a label for the degree of OSA described above.

Of course, the fourth training data may include pairs of input data and output data for the multiple different test-subjects. By the learning using the fourth training data, the fourth model has a function for the relationship between the input data of the fourth training data and the output data of the training data, and expresses the function using various parameters. That is, the fourth model may express the relationship between the input data and the output data of the fourth training data using the parameters of weights and biases.

In summary, the present device 100 may predict the degree of OSA of the subject by using the first and second models, the third model, or the first and fourth models. In this case, the first to fourth models are models trained based on the machine learning technique as described above. That is, the present device 100 may predict the degree of OSA of the subject by performing inference on each required model.

Accordingly, the present device 100 may perform a function of training (hereinafter, referred to as the “first function”) that trains the model by using training data based on the test-subject. In this case, the function of training the first model based on the first training data may be referred to as a “1-1th function”, the function of training the second model based on the second training data may be referred to as a “1-2th function”, the function of training the third model based on the third training data may be referred to as a “1-3th function”, and the function of training the fourth model based on the fourth training data may be referred to as a “1-4th function”.

In addition, the present device 100 may perform a function (hereinafter, referred to as a “second function”) of performing the inference on the model by inputting input data on the subject into the model using the trained model. In this case, the function of performing inference on the first model may be referred to as a “2-1th function”, the function of performing inference on the second model may be referred to as a “2-2th function”, the function of performing inference on the third model may be referred to as a “2-3th function”, and the function of performing inference on the fourth model may be referred to as a “2-4th function”. Depending on the performance of this second function, the present device 100 may predict the degree of OSA for the subject.

Of course, the present device 100 may also operate as a server performing the first or second function, and may transmit the results according to the first or second function to another device.

The present device 100 may be an electronic device capable of computing for the first or second function. For example, the electronic device may be a general-purpose computing system such as a desktop personal computer, a laptop personal computer, a tablet personal computer, a netbook computer, a workstation, a personal digital assistant (PDA), or a smartpad, or an embedded system implemented with embedded Linux or the like, but is not limited thereto.

Referring to FIG. 1, the present device 100 may include the input unit 110, the tranceiver 120, a display 130, the memory 140, and the processor 150. In this case, the memory 140 and the processor 150 may correspond to essential components, and the rest may correspond to additional components. The processor 150 may execute at least one instruction stored in the memory.

The input unit 110 is a configuration that generates input data in response to various user inputs. The input unit 110 may include various input means. For example, the input unit 110 may include at least one of a microphone, a camera, a scanner, a touch pad, a keyboard, a mouse, a mouse pen, or various sensors, but is not limited thereto.

The tranceiver 120 is a configuration that performs communication of various data with other devices. For example, the tranceiver 120 may receive the training data, the machine learning model, various data required for performing the first and second functions, or a program related to the method described below from other devices. In addition, the tranceiver 120 may transmit the result of performing the first or second function to other devices. For example, the tranceiver 120 may perform wireless communication such as 5th generation communication (5G), long term evolution-advanced (LTE-A), long term evolution (LTE), Bluetooth, Bluetooth low energy (BLE), near field communication (NFC), and WiFi communication, or may perform wired communication such as cable communication, but is not limited thereto.

The display 130 is a configuration that outputs output data to the screen. That is, the display 130 may output various output data according to the performance of the first and second functions to the screen. For example, the display 130 may include a liquid crystal display (LCD), a light emitting diode (LED) display, an organic light emitting diode (OLED) display, a micro electro mechanical systems (MEMS) display, or an electronic paper display, but is not limited thereto. In addition, the display 130 may be implemented as a touch screen, or the like, by being combined with the input unit 110.

The memory 140 may store various information necessary for the operation of the electronic device 100. For example, the memory 140 may store the training data, the machine learning model, various data necessary for performing the first and second functions, or a program related to the method to be described later. For example, the memory 140 may include, but is not limited to, a volatile memory device such as DRAM or SRAM, a nonvolatile memory device such as PRAM, MRAM, ReRAM, or NAND flash memory, or a hard disk drive (HDD) or a solid-state drive (SSD). In addition, the memory 140 may be, but is not limited to, a cache, a buffer, a main memory, or an auxiliary memory, or a separately provided storage system, depending on the purpose/location of the memory.

The memory may store information about the first machine learning model, the second machine learning model, the third machine learning model, and the fourth machine learning model. The information about the machine learning model may include at least one of the training data, input data, the algorithm, and learning parameters of the machine learning model.

The processor 150 may perform various control operations of the electronic device 100. That is, the processor 150 may control the performance of the first and second functions and the performance of the method to be described later. In addition, the processor 150 may control the operations of the remaining components of the electronic device 100, such as the input unit 110, the tranceiver 120, the display 130, the memory 140, or the like. For example, the processor 150 may include a hardware processor or a software process executed by the processor, but is not limited thereto. For example, a processor may include, but is not limited to, a microprocessor, a central processing unit (CPU), a processor core, a multiprocessor, an application-specific integrated circuit (ASIC), or a field programmable gate array (FPGA).

Hereinafter, a method according to one embodiment of the present disclosure will be described in more detail.

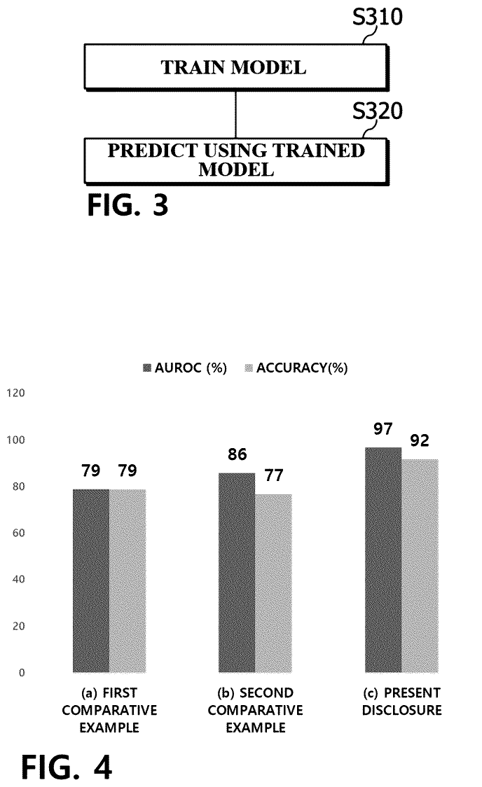

FIG. 3 illustrates a flowchart of a method according to one embodiment of the present disclosure.

A method (hereinafter, referred to as “the present method”) according to one embodiment of the present disclosure may be performed under the control of the processor 150 of the electronic device 100. The present method may include S310 and S320 as a method for performing first and second functions, as illustrated in FIG. 3. That is, S310 is the step for performing the first function, and S320 is the step for performing the second function.

The processing for S310 and S320 may be controlled by the processor 150. Of course, this method may perform only S310, or may perform only S320 using a pre-trained model.

First, in S310, the processor 150 controls to train the model according to the machine learning technique of the supervised learning using the training data.

That is, the processor 150 may control the performance of the first function. Accordingly, the processor 150 may train the first model using the first training data, and may train the second model using the second training data. Alternatively, the processor 150 may train the third model using the third training data. Alternatively, the processor 150 may train the fourth model using the fourth training data.

For example, the machine learning techniques applied to train the model may include, but are not limited to, Artificial neural network, Boosting, Extreme gradient boosting, Bayesian statistics, Logistic regression, Decision tree, Gaussian process regression, Support vector machine, Random forest, Symbolic machine learning, Ensembles of classifiers, or Deep learning.

In particular, the model may be a deep learning model trained by the deep learning technique. In this case, the model expresses the relationship between the input data and output data of the training data as multiple layers, and the multiple representation layers may be also referred to as a “neural network”. That is, the model may express the relationship between the input data and output data of the training data through parameters of multiple hidden layers.

For example, the deep learning techniques may include, but are not limited to, Deep Neural Network (DNN), Convolutional Neural Network (CNN), Recurrent Neural Network (RNN), Restricted Boltzmann Machine (RBM), Deep Belief Network (DBN), and Deep Q-Networks. In particular, since the first model is a model for predicting a class for an input facial photograph, it may be preferable that the first model be a model trained using a CNN-based deep learning technique.

However, in S310, when training the second model, the multiple models trained using different machine learning techniques may be implemented, and then the model with the best performance (for example, accuracy, or the like) among the implemented multiple models may be selected as the second model. This method may be equally applied to the third or fourth models.

However, since the first to fourth models and the first to fourth training data have already been described related to the present device 100, detailed descriptions thereof will be replaced with the corresponding descriptions.

Next, in S320, the processor 150 inputs the subject's input data into the model trained according to S310 and performs the inference on the corresponding model, thereby generating the prediction result for the subject's degree of OSA.

In this case, the processor 150 may perform inference on the first and second models to control the subject's degree of OSA to be predicted. Alternatively, the processor 150 may control the subject's degree of OSA to be predicted using the third model. Alternatively, the processor 150 may control the subject's degree of OSA to be predicted using the first and fourth models.

When performing inference on the first model that has been previously trained, the facial photograph of the subject may be input as the input data to the first model. Accordingly, the first model outputs the probability value of each class for the subject as the output data. In this case, the corresponding probability value of the class with the largest probability value among the probability values of the classes or the processed value of the corresponding probability value may be utilized as the first analysis data included in the input data for inference of the second or fourth model.

When performing inference on the second model that has been previously trained, the response data for the OSA screening questionnaire of the subject and the first analysis data of the subject may be input as input data to the second model, respectively. In this case, the first analysis data of the subject in the corresponding input data is data derived based on the output data according to the inference of the first model. Accordingly, the second model outputs the degree of OSA of the subject according to the corresponding input data as the output data.

When performing inference on the previously trained third model, the subject's response data for the OSA screening questionnaire and the subject's second analysis data may be input as the input data to the third model, respectively. In this case, the subject's second analysis data in the input data includes the distance to the landmark derived from the subject's facial photograph. Accordingly, the third model outputs the subject's degree of OSA according to the input data as the output data.

When performing inference on the previously trained fourth model, the subject's response data for the OSA screening questionnaire, the subject's first analysis data, and the subject's second analysis data may be input as input data to the fourth model, respectively. In this case, the subject's first analysis data in the input data is data derived based on the output data according to the inference of the first model. In addition, the subject's second analysis data in the input data includes the distance to the landmark derived from the subject's facial photograph. Accordingly, the fourth model outputs the degree of OSA of the subject according to the input data as the output data.

Depending on the performance of the inference on the second to fourth models, each probability value for multiple states related to the degree of OSA may be output. Accordingly, the processor 150 may output the state for the highest probability value among the probability values as the final prediction result for the degree of OSA.

For example, depending on the performance of the inference on the second to fourth models, the degree of OSA may output the probability value for each of the four states of the normal, mild degree, moderate degree, and severe degree, or may output the probability value for each of the first and second states.

However, since the response data for the OSA screening questionnaire and the first and second analysis data have already been described in relation to the present device 100, the detailed description thereof will be replaced with the corresponding description.

Meanwhile, the processor 150 may control the display 130 to display the final prediction result for the degree of OSA output from at least one of the second to fourth models according to S320. In particular, the processor 150 may control the display 130 to display a mark or color that is distinguished from the case where another state is finally predicted when the prediction result indicates the degree of OSA of the specific state.

For example, when the degree of OSA corresponding to the moderate degree or the severe degree among the four states of the normal, mild degree, moderate degree, and severe degree is finally predicted, a mark or color different from the case where the degree of OSA of the normal and mild degree is finally predicted may be displayed on the display 130. Alternatively, when the degree of OSA corresponding to the second state of the first and second states is finally predicted, a mark or color different from the case where the degree of OSA of the first state is finally predicted may be displayed on the display 130. Through this, the severity of the corresponding degree of OSA may be emphasized.

In addition, the processor 150 may transmit the final prediction result for the degree of OSA output from at least one of the second to fourth models according to S320 to another device through the tranceiver 120.

Meanwhile, the above-described method may be executed by loading a program into the memory 140 and executing the program under the control of the processor 150. Such a program may be stored in the memory 140 of various types of non-transitory computer-readable media. The non-transitory computer-readable media include various types of tangible storage media.

For example, the non-transitory computer-readable medium includes, but is not limited to, magnetic recording media (for example, flexible disks, magnetic tapes, hard disk drives), magneto-optical recording media (for example, magneto-optical disks), CD-ROMs (read only memories), CD-Rs, CD-R/Ws, or semiconductor memories (for example, mask ROMs, programmable ROMs (PROM), erasable PROMs (EPROMs), flash ROMs, and random access memories (RAMs)).

In addition, the program may be supplied by various types of transitory computer-readable media. For example, the transitory computer-readable medium may include, but is not limited to, electrical signals, optical signals, and electromagnetic waves. That is, the transitory computer-readable medium may supply the program to the processor 150 through wired communication channels such as wires and optical fibers, or wireless communication channels.

### Experiment

Meanwhile, various experiments were performed to compare the performance of the comparative example and the embodiment of the present disclosure. Here, the first and second experiments are experiments on predicting the degree of OSA based on the AHI criterion, while the third experiment is an experiment on predicting the degree of OSA based on the RDI criterion, and these experiments will be described below.

### First Experiment

FIG. 4 illustrates a graph of the results of a first experiment on comparing the performances of one embodiment of the present disclosure and the comparative example.

First, the first comparative example is a technique for diagnosing the OSA using the OSA screening questionnaire based on the first technique. That is, the machine learning model related to the first technique was trained using the machine learning technique based on the supervised learning using training data, and then the performance of the machine learning model was measured. In this regard, the machine learning model according to the first technique using STOP-BANG determines that the OSA risk corresponds to the OSA of the moderate degree or higher when the total score according to the answer to the STOP-BANG is 4 points. In this case, the actual PSG was performed, and the STOP-BANG criterion was applied to the test-subject who was confirmed to have the OSA symptoms according to the AHI criterion. In particular, in the training data for training the machine learning model according to the first technique, the input data is the test-subject's answer according to STOP-BANG, and the output data is two classes of the first and second states for the degree of OSA. In this case, the reference value of STOP-BANG dividing the first and second states was set to 4. Accordingly, the output data of the training data for the case where the STOP-BANG range is less than 4 was set to be the first state, and the output data of the training data for the case where the STOP-BANG range is 4 or more was set to be the second state. The machine learning model according to a first conventional technique was trained based on this training data. As a result, the screening performance of the machine learning model according to the first technique was confirmed to have an Area Under Receiver Operating Characteristic (AUROC or Arear Under ROC) (ROC area) of 79.1% and an accuracy of 79.1%, as illustrated in (a) of FIG. 4.

Next, the second comparative example is based on the second technique, and the machine learning model related to the second technique was trained using the machine learning technique based on the supervised learning using the training data, and then the performance of the machine learning model was measured. In particular, in the training data for training the machine learning model according to the second technique, the input data is the facial photograph of the test-subject, and the output data is two classes of the first and second states for the degree of OSA. In this case, the reference value of the AHI dividing the first and second states was set to 15. Accordingly, the output data of the training data for cases where the AHI range is less than 15 was set to be the first state, and the output data of the training data for cases where the AHI range is 15 or more was set to be the second state. According to this learning, the machine learning model according to the second technique operates as the classification model that predicts the class of the degree of OSA of the subject when the subject's facial photograph is input as the input data. The screening performance of the machine learning model according to the second technique was confirmed to have an AUROC of 85.7% and an accuracy of 76.7%, as illustrated in (b) of FIG. 4.

Meanwhile, in relation to the embodiment of the present disclosure, the first and second models were trained based on the first and second training data, and then the performance of the trained second model was measured. In this case, the OSA screening questionnaire used eight questions of STOP-BANG. That is, the input data for the second model includes response data according to eight questions of STOP-BANG and first analysis data according to the first model. In this case, the output data of the first model may have classes of the first and second states, and the output data of the second model may also have classes of the first and second states. Of course, the AHI reference value that divides the first and second states was set to 15. Accordingly, the output data of the second training data for cases where the AHI range is less than 15 was set to be the first state, and the output data of the second training data for cases where the AHI range is 15 or more was set to be the second state.

The screening performance according to the second model according to the present disclosure was confirmed to have an AUROC of up to 97.2% and an accuracy of up to 91.9%, as illustrated in (c) of FIG. 4 (see Table 3 below). That is, it was confirmed that the second model according to the embodiment of the present disclosure had significantly improved performance compared to the machine learning models according to the first and second techniques.

### Second Experiment

In addition, in relation to the present disclosure, after training the third model based on the third training data, the performance of the trained third model was measured. In this case, the OSA screening questionnaire used the same eight questions of STOP-BANG as in the first experiment. That is, the input data for the third model includes response data according to the eight questions of STOP-BANG and the second analysis data. In this case, the second analysis data is data related to the distance to the landmark in the facial photograph of the test-subject, and 15 distances according to Table 2 were used. The output data of the third model may have classes of the first and second states. Of course, the reference value of the AHI dividing the first and second states was set to 15. Accordingly, the output data of the third training data for cases where the AHI range is less than 15 was set to be the first state, and the output data of the third training data for cases where the AHI range is 15 or more was set to be the second state. The screening performance according to the third model according to the present disclosure was confirmed to have an AUROC of up to 85.6% and an accuracy of up to 82.6% (see Table 3 below). That is, it was confirmed that the third model according to the present disclosure showed superior performance compared to the first prior art according to the first experiment.

Meanwhile, in the first and second experiments, with respect to the second and third models according to the present disclosure, multiple models were trained with four machine learning techniques, respectively, and their respective performances were measured. In this case, the machine learning techniques used were logistic regression (LR), random forest (RF), support vector machine (SVM), and extreme gradient boosting (XGBoost). That is, with respect to the second model, four models according to LR, Rf, SVM, and XGBoost were trained and their performances were measured, and with respect to the third model, four models according to LR, Rf, SVM, and XGBoost were trained and their performances were measured.

In this case, the test-subject was a total of 748 people (n=748), including 213 people (28.5%) in the control group and 535 people (71.5%) with OSA symptoms, and the dataset for them was used. Of course, there are cases where a 1:1 random under sampling (RUS) dataset consisting of 426 test-subjects (n=426) was used, and cases where a 1:2 RUS dataset consisting of 639 test-subjects (n=639) was used.

Accordingly, the performances of various second and third models according to various machine learning techniques (LR, Rf, SVM, and XGBoost) were measured using each of these datasets, and the details of the results are as illustrated in Table 3 below. In particular, the performances of the second and third models according to LR were confirmed to be superior to the second and third models according to other Rf, SVM, and XGBoost.

TABLE 3

| | | Machine | | | | | | | | F1 | |
|---|---|---|---|---|---|---|---|---|---|---|---|
| | Model | learning | AUROC | AUPRC | Sensitivity | Specificity | Accuracy | PPV | NPV | score | Threshold |
| Dataset | type | technique | (%) | (%) | (%) | (%) | (%) | (%) | (%) | (%) | (%) |
| Original | Second | LR | 93.9 | 91.2 | 91.4 | 86.1 | 92.0 | 94.4 | 86.1 | 90.4 | 57.8 |
| n = 748 | model | RF | 95.2 | 97.8 | 91.6 | 86.1 | 90.0 | 94.2 | 80.4 | 92.9 | 64.9 |
| | | SVM | 95.5 | 98.0 | 93.5 | 86.1 | 91.3 | 94.3 | 84.1 | 93.9 | 52.3 |
| | | XGBoost | 93.7 | 96.0 | 94.4 | 83.7 | 91.3 | 93.5 | 85.7 | 94.0 | 47.9 |
| | Third | LR | 86.7 | 93.5 | 79.4 | 86.0 | 81.3 | 93.4 | 62.7 | 85.9 | 66.4 |
| | model | RF | 81.6 | 89.5 | 63.6 | 90.7 | 71.3 | 94.4 | 50.0 | 76.0 | 75.8 |
| | | SVM | 85.8 | 93.1 | 73.8 | 90.7 | 78.7 | 95.2 | 58.2 | 83.2 | 73.6 |
| | | XGBoost | 83.1 | 90.7 | 72.0 | 86.0 | 76.0 | 92.8 | 55.2 | 81.1 | 64.8 |
| RUS 1:1 | Second | LR | 97.2 | 97.0 | 93.0 | 90.7 | 91.9 | 90.9 | 92.9 | 92.0 | 54.7 |
| n = 426 | model | RF | 96.0 | 95.3 | 97.7 | 83.7 | 90.7 | 85.7 | 97.3 | 91.3 | 30.8 |
| | | SVM | 91.6 | 91.9 | 90.7 | 76.7 | 83.7 | 79.6 | 89.2 | 84.8 | 29.8 |
| | | XGBoost | 96.7 | 95.7 | 95.3 | 86.0 | 90.7 | 87.2 | 94.9 | 91.1 | 39.6 |
| | Third | LR | 85.6 | 84.8 | 74.4 | 90.7 | 82.6 | 88.9 | 78.0 | 81.0 | 54.8 |
| | model | RF | 77.3 | 77.1 | 74.4 | 76.7 | 75.6 | 76.2 | 75.0 | 75.3 | 49.6 |
| | | SVM | 77.9 | 74.6 | 81.4 | 69.8 | 75.6 | 72.9 | 78.9 | 76.9 | 29.8 |
| | | XGBoost | 78.3 | 77.1 | 67.4 | 81.4 | 74.4 | 78.4 | 71.4 | 72.5 | 50.0 |
| RUS 1:1 | Second | LR | 91.5 | 95.2 | 90.6 | 79.1 | 86.7 | 89.5 | 81.0 | 90.1 | 57.2 |
| n = 639 | model | RF | 90.5 | 94.5 | 91.8 | 76.7 | 86.7 | 88.6 | 82.5 | 90.2 | 56.8 |
| | | SVM | 90.8 | 94.7 | 96.5 | 67.4 | 86.7 | 85.4 | 90.6 | 90.6 | 41.3 |
| | | XGBoost | 91.9 | 95.6 | 77.6 | 88.4 | 81.3 | 93.0 | 66.7 | 84.6 | 73.4 |
| | Third | LR | 82.5 | 87.9 | 80.0 | 74.4 | 78.1 | 86.1 | 65.3 | 82.9 | 60.9 |
| | model | RF | 76.4 | 83.9 | 58.8 | 79.1 | 65.6 | 84.7 | 49.3 | 69.4 | 70.4 |
| | | SVM | 70.8 | 76.6 | 92.9 | 46.5 | 77.3 | 77.5 | 76.9 | 84.5 | 47.4 |
| | | XGBoost | 81.8 | 86.9 | 85.9 | 62.8 | 78.1 | 82.0 | 69.2 | 83.9 | 56.3 |

### Experiment 3

Meanwhile, unlike the experiment related to Table 3, based on the RDI criterion, multiple models related to the second model according to the present disclosure were trained with four machine learning techniques, and their respective performances were measured. In this case, the machine learning techniques used were LR, RF, SVM, and XGBoost, the same as in the second experiment. That is, with respect to the second model, four models according to LR, Rf, SVM, and XGBoost were trained and their performances were measured. In particular, the classes for the degree of OSA were two classes, the first and second states. In this case, the reference value of RDI dividing the first and second states was set to 15. Accordingly, the output data of the training data for cases where the RDI range is less than 15 was set as the first state, and the output data of the training data for cases where the RDI range is 15 or more was set as the second state.

In this case, the test-subject was a total of 748 people (n=748), including 174 people (23.3%) in the control group and 574 people (76.7%) with OSA symptoms, and a 1:1 random under sampling (RUS) dataset consisting of 348 test-subjects (n=348) of 748 people was used.

Accordingly, the performances of various second models according to various machine learning techniques (LR, Rf, SVM, and XGBoost) were measured using each of these datasets, and it was confirmed that the maximum AUROC was 94.1% and the accuracy was 91.6%, and the details of the results are as illustrated in Table 4 below. In particular, the performance of the second model according to LR was confirmed to be superior to the second models according to other Rf, SVM, and XGBoost.

TABLE 4

| | | Machine | | | | | | | | F1 | |
|---|---|---|---|---|---|---|---|---|---|---|---|
| | Model | learning | AUROC | AUPRC | Sensitivity | Specificity | Accuracy | PPV | NPV | score | Threshold |
| Dataset | type | technique | (%) | (%) | (%) | (%) | (%) | (%) | (%) | (%) | (%) |
| RUS 1:1 | Second | LR | 94.1 | 91.6 | 91.4 | 82.9 | 88.1 | 85.2 | 90.6 | 89.7 | 52.3 |
| n = 348 | model | RF | 90.7 | 88 | 68.5 | 97.1 | 82.8 | 96 | 75.5 | 80 | 67.7 |
| | | SVM | 94.5 | 95.1 | 80 | 97.1 | 88.5 | 96.5 | 81.9 | 87.5 | 69.8 |
| | | XGBoost | 91.6 | 92 | 77.1 | 91.4 | 84.2 | 90 | 80 | 83 | 67 |

According to the present disclosure configured as described above, the degree of OSA of the subject is predicted by comprehensively reflecting the response data of the subject to the OSA screening questionnaire and the analysis results of a facial photograph containing anatomical information, and thus, it is possible to increase the accuracy of the prediction. That is, the present disclosure predicts the degree of OSA of the subject by comprehensively using the first and second prior arts, and thus has the advantage of complementing the respective problems of the first and second prior arts. In addition, according to the present disclosure, the degree of OSA of the subject is predicted by using the second machine learning model trained based on the response data to the OSA screening questionnaire and the analysis results of the first machine learning model for the facial photograph, respectively, and thus, it is possible to predict the OSA information of the subject very accurately. Moreover, according to the present disclosure, the degree of OSA of the subject is predicted by using the third machine learning model trained based on the response data to the OSA screening questionnaire and the distance to the landmark of the facial photograph, respectively, and thus, it is possible to predict the OSA information of the subject very accurately.

In the detailed description of the present disclosure, specific embodiments have been described, but it is obvious that various modifications are possible within the scope of the present disclosure. Therefore, the scope of the present disclosure is not limited to the described embodiments, but should be determined by the claims and equivalents thereof.

What is claimed is:

1. A method for predicting a degree of obstructive sleep apnea (OSA) by executing at least one instruction stored in a memory by a processor, the method comprising:
generating analysis data from facial photograph information of an analysis subject;
storing response data of an OSA screening questionnaire of the analysis subject in the memory;
inputting the analysis data and the response data into a pre-trained machine learning model and inferring information about the degree of OSA; and
transmitting the inference result to at least one terminal or outputting the inference result to a display.

2. The method of claim 1, wherein the generating of the analysis data includes
inputting the facial photograph information into a first machine learning model, and
generating first analysis data inferring the degree of OSA by the first machine learning model.

3. The method of claim 2, wherein the inferring of the information about the degree of OSA includes
inputting the first analysis data and the response data into a second machine learning model, and
inferring the information about the degree of OSA by the second machine learning model and generating the inference result.

4. The method of claim 1, wherein the generating of the analysis data includes
extracting a plurality of landmark information from the facial photograph information, and
generating second analysis data, which is distance information between the landmarks, by using the plurality of landmark information.

5. The method of claim 4, wherein the inferring of the information about the degree of OSA includes
inputting the second analysis data and the response data into a third machine learning model, and
inferring the information about the degree of OSA by the third machine learning model and generating the inference result.

6. The method of claim 1, wherein the generating of the analysis data includes
inputting the facial photograph information into a first machine learning model,
generating first analysis data inferring the degree of OSA by the first machine learning model,
extracting a plurality of landmark information from the facial photograph information, and
generating second analysis data, which is distance information between the landmarks, using the plurality of landmark information, and
the inferring of the information about the degree of OSA includes
inputting the first analysis data, the second analysis data, and the response data into a fourth machine learning model, and
inferring the information about the degree of OSA by the fourth machine learning model and generating the inference result.

7. The method of claim 1, wherein the machine learning model infers information about a plurality of classes based on a preset range for an apnea-hypopnea index or a respiratory distress index.

8. An apparatus for predicting obstructive sleep apnea, the apparatus comprising:
a memory that stores at least one instruction;
a processor that executes the at least one instruction,
wherein the processor
generates analysis data from facial photograph information of an analysis subject,
stores response data of an OSA screening questionnaire of the analysis subject in the memory,
inputs the analysis data and the response data into a pre-trained machine learning model and infers information about the degree of OSA, and
transmits the inference result to at least one terminal or outputs the inference result to a display.

9. The apparatus of claim 8, wherein the processor
inputs the facial photograph information into a first machine learning model, and
generates first analysis data inferring the degree of OSA by the first machine learning model to generate the analysis data.

10. The apparatus of claim 9, wherein the processor
inputs the first analysis data and the response data into a second machine learning model, and
infers the information about the degree of OSA by the second machine learning model and generates the inference result to infer the information about the degree of OSA.

11. The apparatus of claim 8, wherein the processor
extracts a plurality of landmark information from the facial photograph information, and
generates second analysis data, which is distance information between the landmarks, by using the plurality of landmark information to generate the analysis data.

12. The apparatus of claim 11, wherein the processor
inputs the second analysis data and the response data into a third machine learning model, and
infers the information about the degree of OSA by the third machine learning model to infer the information about the degree of OSA.

13. The apparatus of claim 8, wherein the processor
inputs the facial photograph information into a first machine learning model,
generates first analysis data inferring the degree of OSA by the first machine learning model,
extracts a plurality of landmark information from the facial photograph information,
generates second analysis data, which is distance information between the landmarks, using the plurality of landmark information, to generate the analysis data,
inputs the first analysis data, the second analysis data, and the response data into a fourth machine learning model, and
infers the information about the degree of OSA by the fourth machine learning model and generates the inference result to infer the information about the degree of OSA.

14. The apparatus of claim 8, wherein the machine learning model infers information about a plurality of classes based on a preset range for an apnea-hypopnea index or a respiratory distress index.
